(19) United States
(12) Patent Application Publication
(10) Pub. No.: US 2011/0128359 A1
(43) Pub. Date: Jun. 2, 2011

(54) IMAGING APPARATUS, IMAGING SYSTEM, METHOD OF CONTROLLING THE APPARATUS AND THE SYSTEM, AND PROGRAM

(75) Inventors: Keigo Yokoyama, Honjo-shi (JP); Tadao Endo, Honjo-shi (JP); Toshio Kameshima, Kumagaya-shi (JP); Tomoyuki Yagi, Honjo-shi (JP); Katsuro Takenaka, Honjo-shi (JP); Sho Sato, Kumagaya-shi (JP)

(73) Assignee: CANON KABUSHIKI KAISHA, Tokyo (JP)

(21) Appl. No.: 12/950,868

(22) Filed: Nov. 19, 2010

(30) Foreign Application Priority Data
Dec. 1, 2009 (JP) PCT/JP2009/070201

(51) Int. Cl. H04N 7/18

(52) U.S. Cl. 348/61; 348/E07.085

(57) ABSTRACT

An imaging apparatus includes a detector including multiple pixels arranged in a matrix. The detector performs an image capturing operation whereby light or radiation incident on the pixels is converted into an image signal. The imaging apparatus also includes a bias light source and a control unit. The image capturing operation includes a first image capturing operation in which the detector is scanned in a scanning area A and a second image capturing operation in which the detector is scanned in a scanning area B larger than the scanning area A. The control unit controls the bias light source to emit bias light on the basis of a control signal indicative of an amount of integration of accumulation times in the first image capturing operation during a period between the first and second image capturing operations in accordance with switching from an irradiation field A to an irradiation field B.

### TECHNICAL FIELD

The present invention relates to an imaging apparatus, an imaging system, a method of controlling the apparatus and the system, and a program for implementing the method into the apparatus or the system. More specifically, the present invention relates to a radiation imaging apparatus, a radiation imaging system, a method of controlling the apparatus and the system, and a program. The foregoing are preferably used in capturing of still images, such as photography, and recording of movies, such as fluoroscopy, for medical diagnosis.

### BACKGROUND ART

In recent years, radiation imaging apparatuses using flat panel detectors (hereinafter abbreviated as FPDs) made of semiconductor materials have come into practical use as image capturing apparatuses used in medical image diagnosis and non-destructive tests using X rays. Such radiation imaging apparatuses are used as digital imaging apparatuses for capturing of still image, such as photography, and recording of movies, such as fluoroscopy, for example, in the medial image diagnosis.

Arbitrary switching of the areas (field-of-view sizes) where the readout by the FPDs is performed is discussed in such a radiation imaging apparatus, as disclosed in Patent Literatures 1 and 2.

### CITATION LIST

### Patent Literature

PTL 1 Japanese Patent Laid-Open No. 11-128213
PTL 2 Japanese Patent Laid-Open No. 11-318877

However, when the readout areas are expanded as the result of the switching, the areas where the scanning by the FPD is performed differ from the areas where the scanning by the FPD is not performed in the sensitivity of pixels and/or the dark time outputs. Accordingly, ghost (difference in level) affected by the readout area (scanning area) can occur in an image that is acquired to cause a reduction in image quality.

### SUMMARY OF INVENTION

Embodiments of the present invention describe an imaging apparatus and an imaging system capable of reducing the difference in level that can occur in an acquired image when switching the scanning area. Advantageously, the disclosed imaging apparatus and system including the apparatus can significantly improve image quality.

An imaging system according to the present invention includes an imaging apparatus and a control computer that controls the imaging apparatus. The imaging apparatus includes a detector in which a plurality of pixels each including a conversion element that converts radiation or light into an electric charge are arranged in a matrix and which performs an image capturing operation to output image data corresponding to radiation or light that is emitted; a bias light source that irradiates the detector with bias light different from the radiation or the light; and a control unit that controls operations including the image capturing operation of the detector and an operation of the bias light source. The image capturing operation includes a first image capturing operation in which the detector is scanned in a first scanning area corresponding to part of the plurality of pixels to output image data in the first scanning area and a second image capturing operation in which the detector is scanned in a second scanning area larger than the first scanning area to output image data in the second scanning area. The control computer determines the operation of the bias light source on the basis of information about the amount of integration of accumulation times in the first image capturing operation and supplies a control signal based on the determined operation of the bias light source to the control unit. The control unit controls the operation of the bias light source so that the bias light source emits the bias light on the basis of the control signal during a period between the first image capturing operation and the second image capturing operation in accordance with switching from the first scanning area to the second scanning area.

An imaging apparatus according to the present invention includes a detector in which a plurality of pixels each including a conversion element that converts radiation or light into an electric charge are arranged in a matrix and which performs an image capturing operation to output image data corresponding to radiation or light that is emitted; a bias light source that irradiates the pixels with bias light different from the radiation or the light; and a control unit that controls operations including the image capturing operation of the detector and an operation of the bias light source. The image capturing operation includes a first image capturing operation in which the detector is scanned in a first scanning area corresponding to part of the plurality of pixels to output image data in the first scanning area and a second image capturing operation in which the detector is scanned in a second scanning area larger than the first scanning area to output image data in the second scanning area. The control unit controls the operation of the bias light source so that the bias light source emits the bias light on the basis of a control signal based on information about the amount of integration of accumulation times in the first image capturing operation during a period between the first image capturing operation and the second image capturing operation in accordance with switching from the first scanning area to the second scanning area.

A control method according to the present invention is used to control an imaging apparatus that includes a detector in which a plurality of pixels each including a conversion element that converts radiation or light into an electric charge are arranged in a matrix and which performs an image capturing operation to output image data corresponding to radiation or light that is emitted and a bias light source irradiating the pixels with bias light different from the radiation or the light and that controls operations including the image capturing operation of the detector and an operation of the bias light source. The method includes the steps of performing a first image capturing operation in which the detector is scanned in a first scanning area corresponding to part of the plurality of pixels to output image data in the first scanning area; determining the operation of the bias light source on the basis of information about the amount of integration of accumulation times in the first image capturing operation; and emitting the bias light on the basis of the determined operation of the bias light source during a period between the first image capturing operation and a second image capturing operation in which the detector is scanned in a second scanning area larger than the first scanning area to output image data in the second scanning area in accordance with an instruction to switch from the first scanning area to the second scanning area in order to perform the second image capturing operation.

A program according to the present invention causes a computer to control an imaging apparatus that includes a detector in which a plurality of pixels each including a conversion element that converts radiation or light into an electric charge are arranged in a matrix and which performs an image capturing operation to output image data corresponding to radiation or light that is emitted and a bias light source irradiating the pixels with bias light different from the radiation or the light and that controls operations including the image capturing operation of the detector and an operation of the bias light source. The program includes the steps of performing a first image capturing operation in which the detector is scanned in a first scanning area corresponding to part of the plurality of pixels to output image data in the first scanning area; determining the operation of the bias light source on the basis of information about the amount of integration of accumulation times in the first image capturing operation; and emitting the bias light on the basis of the determined operation of the bias light source during a period between the first image capturing operation and a second image capturing operation in which the detector is scanned in a second scanning area larger than the first scanning area to output image data in the second scanning area in accordance with an instruction to switch from the first scanning area to the second scanning area in order to perform the second image capturing operation.

Further features of the present invention will become apparent from the following description of exemplary embodiments with reference to the attached drawings.

### BRIEF DESCRIPTION OF DRAWINGS

FIG. 1 is a conceptual block diagram of an imaging system including an imaging apparatus according to the present invention.

FIG. 2 is a conceptual equivalent circuit diagram of the imaging apparatus according to an embodiment of the present invention.

FIG. 3 is a flowchart showing the operation of the imaging apparatus and the imaging system according to the present invention.

FIG. 4A is a timing chart illustrating the operation of the imaging apparatus and the imaging system of the present invention.

FIG. 4B is a timing chart illustrating the operation of the imaging apparatus and the imaging system of the present invention.

FIG. 4C is a timing chart illustrating the operation of the imaging apparatus and the imaging system of the present invention.

FIG. 4D is a timing chart illustrating the operation of the imaging apparatus and the imaging system of the present invention.

FIG. 5A is a conceptual diagram illustrating a configuration in which a processing operation of the present invention is performed.

FIG. 5B is a timing chart illustrating an imaging operation in accordance with an embodiment of the present invention.

FIG. 5C is a timing chart illustrating a timing configuration in which an imaging operation in accordance with an embodiment of the present invention is performed.

FIGS. 6A and 6B are conceptual equivalent circuit diagrams of an imaging apparatus according to another embodiment of the present invention.

FIG. 7A is a timing chart illustrating an operation of the other imaging apparatus and an imaging system according the present invention.

FIG. 7B is a timing chart illustrating an operation of the other imaging apparatus and the imaging system according the present invention.

FIG. 7C is a timing chart illustrating an operation of the other imaging apparatus and the imaging system according the present invention.

### DESCRIPTION OF EMBODIMENTS

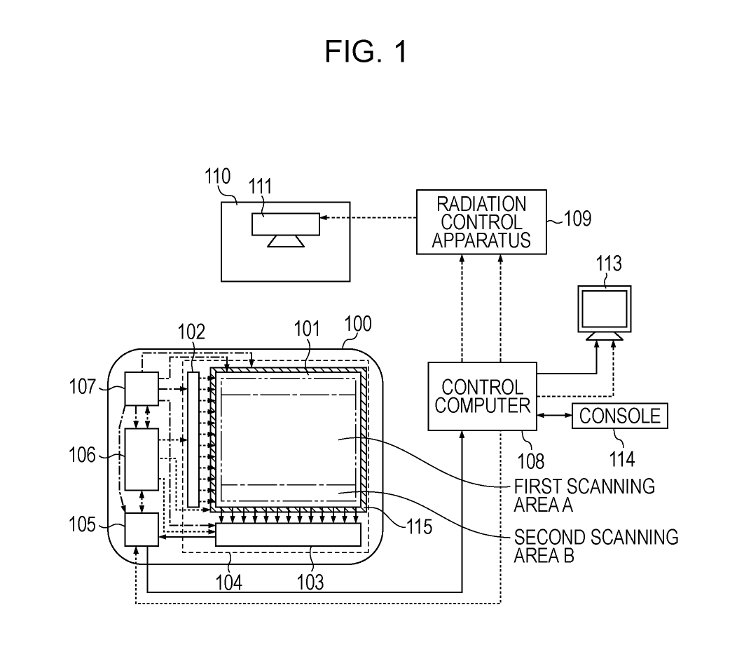

Preferred embodiments of the present invention will herein be described in detail with reference to the attached drawings. A radiation imaging system according to an embodiment shown in FIG. 1 includes an imaging apparatus 100, a control computer 108, a radiation control apparatus 109, a radiation generating apparatus 110, a display apparatus 113, and a console 114. The imaging apparatus 100 includes an FPD 104 including a detection unit 101, a drive circuit 102, and a readout circuit 103. The detection unit 101 includes multiple pixels arranged in a matrix of n lines by m columns; each pixel converts radiation or light incident thereupon into an electrical signal. The drive circuit 102 drives the detection unit 101. The readout circuit 103 receives the electric signal from the detection unit 101 and outputs the electrical signal supplied from the detection unit 101 that is driven as image data. The imaging apparatus 100 also includes a signal processing unit 105 that processes the image data supplied from the FPD (flat panel detector) 104 to output the image data subjected to the processing and a control unit 106 that supplies a control signal to each component to control the operations of the FPD 104 and a bias light source 115 described below. The imaging apparatus 100 further includes a power supply unit 107 that supplies a bias voltage to each component of the imaging apparatus 100, including the bias light source 115. In the imaging apparatus 100, the bias light source 115 irradiates the FPD 104 with bias light. The bias light is emitted separately from the radiation generated by a radiation source 111 described below or the light converted from the radiation by a wavelength converter described below. The signal processing unit 105 receives a control signal from an external control computer 108, and supplies the received control signal to the control unit 106. The control unit 106 controls the drive circuit 102 so that switching between at least two scanning areas of the detection unit 101 is performed in response to the control signal received from the control computer 108. Accordingly, the drive circuit 102 is configured to alternately drive the scanning areas A or B in response to the control signal received from the control unit 106. In other words, the control unit 106 has a function of switching between a first scanning area A and a second scanning area B; and the drive circuit 102 has a function of scanning alternately the scanning area A or scanning area B. In the first scanning area A, part of the multiple pixels (first pixels) is scanned by the drive circuit 102. For example, when the total number of pixels in the detection unit 101 is equal to 2,800 lines×2,800 columns, an area of pixels of 1,000 lines×2,800 columns may be scanned in a first instance by the drive circuit 102. In the second scanning area B, pixels (second pixels) within an area larger than the first scanning area A, for example, an area including all of the pixels in the detection unit 101 is scanned by the drive circuit 102. It should be noted however that, as long as one scanning area is different in size than the other scanning area, it may not be necessary that all of the pixels in the detection unit 101 be scanned. That is, there may be instances in which a valid image can be obtained even if only part of the pixels in the detection unit 101 is included in the first and second scanning areas. In addition, although only two scanning areas have been shown for ease of illustration, it is within the scope of the present invention that the detection unit 101 may be divided into a number of scanning areas greater than two; and that the drive circuit 102 may be configured to alternately drive and scan a number of scanning areas greater than two.

The power supply unit 107 includes a power supply circuit, such as a regulator or an inverter, which receives a voltage from an external power supply or a built-in battery (not shown) to supply a voltage necessary to operate the detection unit 101, the drive circuit 102, the readout circuit 103, and the bias light source 115. The bias light source 115 is provided so as to be opposed to a face (rear face) opposite a light receiving face on which the pixels are provided of a substrate on which the detection unit 101 is provided. The bias light source 115 is arranged so that the entire detection unit 101 is irradiated with the bias light from the rear face. The bias light source 115 is arranged so that an area that is equal to or larger than the second scanning area B of the detection unit 101 can be irradiated with the bias light.

The control computer 108 performs synchronization between the radiation generating apparatus 110 and the imaging apparatus 100. More specifically, the control computer 108 controls transmission of control signals for determining the state of the imaging apparatus 100 and performs image processing for correcting, storing, and/or displaying the image data from the imaging apparatus 100. In addition, the control computer 108 transmits control signals for determining irradiation conditions of the radiation emitted from radiation source 111 on the basis of information received from the console 114 to the radiation control apparatus 109.

The radiation control apparatus 109 controls an operation to emit the radiation from the radiation source 111 included in the radiation generating apparatus 110 in response to the control signals received from the control computer 108. An irradiation field limiting mechanism 112 has a function of changing a certain irradiation field which is irradiated with the radiation or the light corresponding to the radiation and which is in the detection unit 101 in the FPD 104. The console 114 is used to input information about a subject to be imaged and the image capturing conditions for imaging the subject. The subject information and image capturing conditions input through the console 114 are used as control parameters in the control computer 108. Accordingly, the console 114 transmits the subject information and the image capturing conditions to the control computer 108. The display apparatus 113 displays the image data subjected to the image processing in the control computer 108.

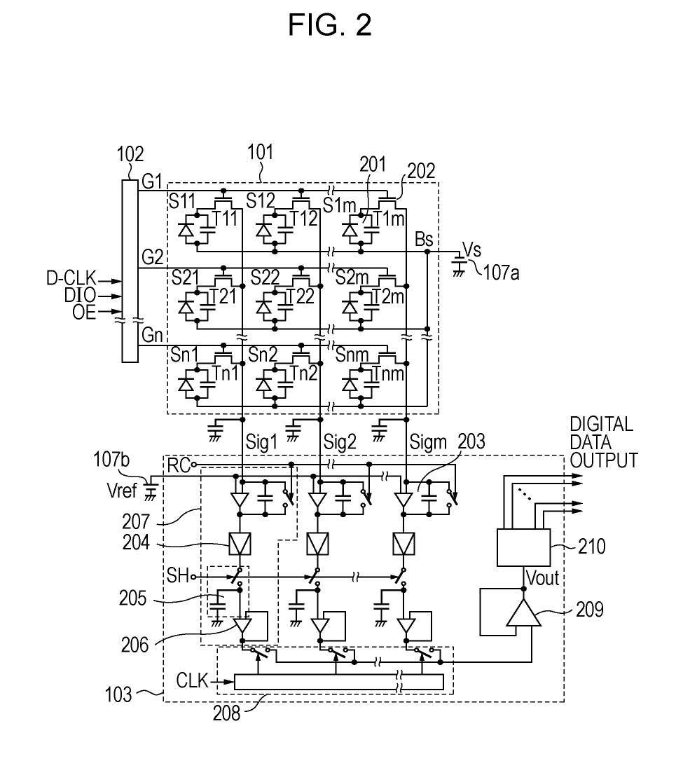

Next, the imaging apparatus according to a first embodiment of the present invention will be described in more detail with reference to FIG. 2. The same reference numerals are used in FIG. 2 to identify the same components shown in FIG. 1. A detailed description of such components is omitted herein. The imaging apparatus in FIG. 2 includes a detailed illustration of the detection unit 101 and the readout circuit 103. As illustrated, the detection unit 101 includes multiple pixels arranged in a matrix of n lines×m columns (n=m=3 is illustrated for convenience), where each of n and m is an integer that is equal to or larger than two. For example, a 17-inch imaging apparatus includes pixels of about 2,800 lines×about 2,800 columns.

The detection unit 101 includes the multiple pixels that are arranged in a matrix of n lines by m columns. Each pixel includes a conversion element 201 that converts radiation or light incident thereupon into an electric charge and a switch element 202 that outputs an electrical signal corresponding to the electric charge. In the present embodiment, a PIN photodiode that is arranged on an insulating substrate, such as a glass substrate, and that is mainly made of an amorphous silicon material is used as a photoelectric transducer for converting the light with which the conversion element is irradiated into the electric charge. An indirect conversion element provided with the wavelength converter at the incident side of the radiation of the above photoelectric transducer or a direct conversion element directly converting the radiation into the electric charge is preferably used as the conversion element. The wavelength converter converts radiation into light within a waveband that can be detected by the photoelectric transducer. A transistor having a control terminal and two main terminals is preferably used as the switch element 202. A thin film transistor (TFT) is used as the switch element 202 in the present embodiment. One electrode of the conversion element 201 is electrically connected to one of the two main terminals of the switch element 202 and the other electrode of the conversion element 201 is electrically connected to a bias power supply 107a via a common bias line Bs. The control terminals of the multiple switch elements in the line direction, for example, the switch elements T11 to Tim are commonly electrically connected to a first-line drive line G1. A drive signal for controlling the conductive state of the switch element 202 is supplied from the drive circuit 102 to the switch elements in each line through the drive line. The drive circuit 102 controls the conductive state and the non-conductive state of the switch elements 202 for every line to scan the pixels for every line. The scanning area of the present invention means an area where the drive circuit 102 scans the pixels for every line in the above manner. Although the pixels of n lines×m columns (where n=m=3) are shown in FIG. 2 for convenience, the pixels of about 1,000 lines×about 2,800 columns are practically scanned by the drive circuit 102 as the first scanning area A when the total number of pixels is equal to, for example, about 2,800 lines×about 2,800 columns. The remaining main terminal of each of the multiple switch elements in the column direction, for example, the switch elements T11 to Tn1 are electrically connected to a first-column signal line Sig1. The electrical signal corresponding to the electric charge of the conversion element is supplied to the readout circuit 103 through the signal line while the switch element is in the conductive state. The electrical signals output from the multiple pixels are transmitted to the readout circuit 103 in parallel through the multiple signal lines Sig1 to Sigm arranged in the column direction.

The readout circuit 103 includes an amplifier circuit 207 for every signal line. The amplifier circuit 207 amplifies each of the electrical signals output in parallel from the detection unit 101. The amplifier circuit 207 includes an integration amplifier 203 that amplifies the output electrical signal, a variable amplifier 204 that amplifies the electrical signal from the integration amplifier 203, a sample-and-hold circuit 205 that samples and holds the amplified electrical signal, and a buffer amplifier 206. The integration amplifier 203 includes an operational amplifier that amplifies the readout electrical signal and outputs the amplified electrical signal, an integration capacitor, and a reset switch. The integration amplifier 203 is capable of varying the value of the integration capacitor to change the gain. The output electrical signal is input into an inverting input terminal of the operational amplifier, a reference voltage Vref is supplied from a reference power supply 107b to a non-inverting input terminal of the operational amplifier, and the amplified electrical signal is output from an output terminal of the operational amplifier. The integration capacitor is arranged between the inverting input terminal and the output terminal of the operational amplifier. The sample-and-hold circuit 205 is provided for every amplifier circuit and includes a sampling switch and a sampling capacitor. The readout circuit 103 further includes a multiplexer 208 that sequentially outputs the electrical signals read out in parallel from the amplifier circuits 207 as a serial image signal and a buffer amplifier 209 that performs impedance conversion to the image signal to output the image signal subjected to the impedance conversion. An analog image signal Vout output from the buffer amplifier 209 is converted into digital image data in an analog-to-digital (A/D) converter 210 and the digital image data is supplied to the signal processing unit 105. The image data processed in the signal processing unit 105 in FIG. 1 is transmitted to the control computer 108.

The drive circuit 102 supplies a drive signal including a conductive voltage Vcom setting the switch element 202 to the conductive state and a non-conductive voltage Vss setting the switch element to the non-conductive state to each drive line in response to a control signal (D-CLK, OE, or DIO) supplied from the control unit 106 in FIG. 1. The drive circuit 102 controls the conductive state and the non-conductive state of the switch element 202 with the control signal to drive the detection unit 101.

The power supply unit 107 in FIG. 1 includes the bias power supply 107a and the reference power supply 107b for the amplifier circuit 207 shown in FIG. 2. The bias power supply 107a supplies a bias voltage Vs to the other electrode of each conversion element through the bias line Bs. The reference power supply 107b supplies the reference voltage Vref to the non-inverting input terminal of each operational amplifier. The power supply unit 107 in FIG. 1 further includes a power supply circuit for the bias light source, such as an inverter, which supplies a voltage necessary for the operation of the bias light source 115.

The control unit 106 in FIG. 1 receives the control signals from the control computer 108, etc. outside the imaging apparatus through the signal processing unit 105 and supplies the various control signals to the drive circuit 102, the power supply unit 107, and the readout circuit 103 to control the operations of the FPD 104 and the bias light source 115. The control unit 106 supplies the control signal D-CLK, the control signal OE, and the control signal DIO to the drive circuit 102 to control the operation of the drive circuit 102. The control signal D-CLK is a shift clock for a shift register used as the drive circuit, the control signal DIO is a pulse transferred by the shift register, and the OE is used to control the output end of the shift register. The control unit 106 is capable of controlling the drive circuit 102 with these control signals to switch between the first scanning area A and the second scanning area B. In addition, the control unit 106 supplies a control signal RC, a control signal SH, and a control signal CLK to the readout circuit 103 to control the operation of each component in the readout circuit 103. The control signal RC is used to control the operation of the reset switch in the integration amplifier, the control signal SH is used to control the operation of the sample-and-hold circuit 205, and the control signal CLK is used to control the operation of the multiplexer 208.

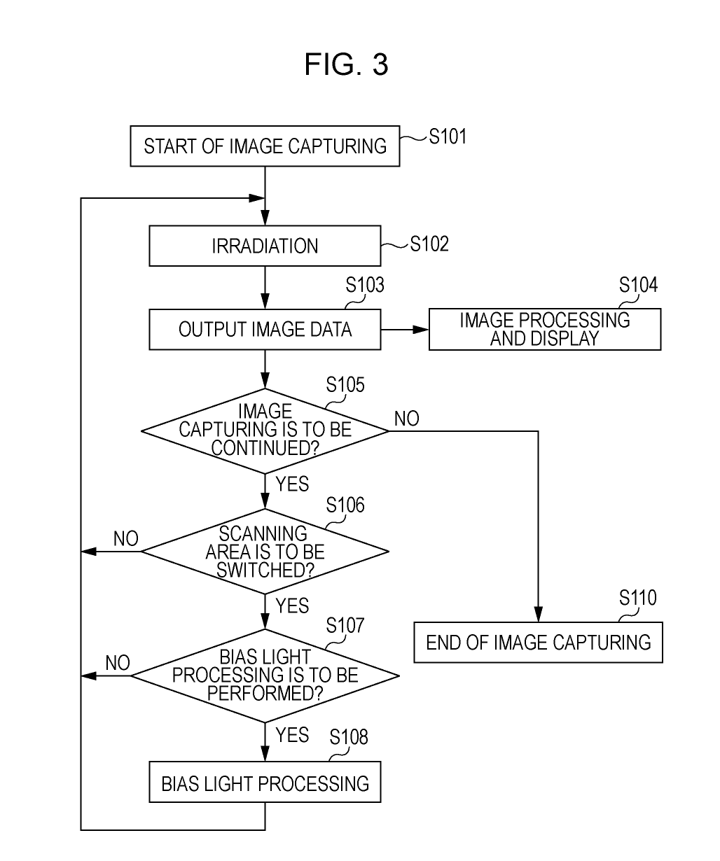

Next, the entire operation of the imaging apparatus and the imaging system of the present invention will be described with reference to FIGS. 1 to 3, in particular with reference to FIG. 3. FIG. 3 illustrates an exemplary process for an imaging operation in accordance with at least one embodiment of the present invention. In FIG. 3, after the irradiation conditions are determined by the control computer 108 in response to an operation by an operator with the console 114, the image capturing is started at step S101. At step S102, an object is irradiated with desired radiation emitted from the radiation generating apparatus 110 controlled by the radiation control apparatus 109 under the determined irradiation conditions. At step S103, the imaging apparatus 100 outputs image data corresponding to the radiation transmitted through the object. The output image data is subjected to the image processing in the control computer 108 and is displayed in the display apparatus 113, at step S104.

At the same time that image data is displayed in the display apparatus 113, the control computer 108 prompts the operator whether the image capturing is to be continued (step S105). If an instruction not to continue the image capturing is received from the operator (NO in S105), the image capturing is terminated at step S110. If an instruction to continue the image capturing is received from the operator (YES in S105), the control computer 108 proceeds to the imaging operation, and at step S106 prompts the operator whether the scanning area is to be switched. If an instruction not to switch the scanning area is received from the operator (NO in S106), the control computer 108 controls the radiation control apparatus 109 and the radiation generating apparatus 110 under the image capturing conditions that have been determined to irradiate the object with the radiation again under the same conditions. If an instruction to switch the scanning area is received from the operator (YES in S106), the control computer 108 continues the imaging operation and determines the scanning area to be switched to. After switching to the desired scanning area, at step S107, the control computer 108 determines whether a bias light processing operation is to be performed. If the control computer 108 determines that the bias light processing operation is to be performed (YES in S107), the process advances to step S108, where the control computer 108 supplies the control signal to the control unit 106 so as to cause the imaging apparatus 100 to perform the bias light processing operation described in detail below. After the imaging apparatus 100 completes the bias light processing operation, the process returns to step S102 where the control computer 108 controls the radiation control apparatus 109 and the radiation generating apparatus 110 so that the radiation is emitted in the image capturing operation after the scanning area is switched. In addition, the control computer 108 supplies the control signal to the control unit 106 so that the image capturing after the scanning area is switched is performed. The imaging apparatus 100 performs the next image capturing in the scanning area after the switching in response to the control signal.

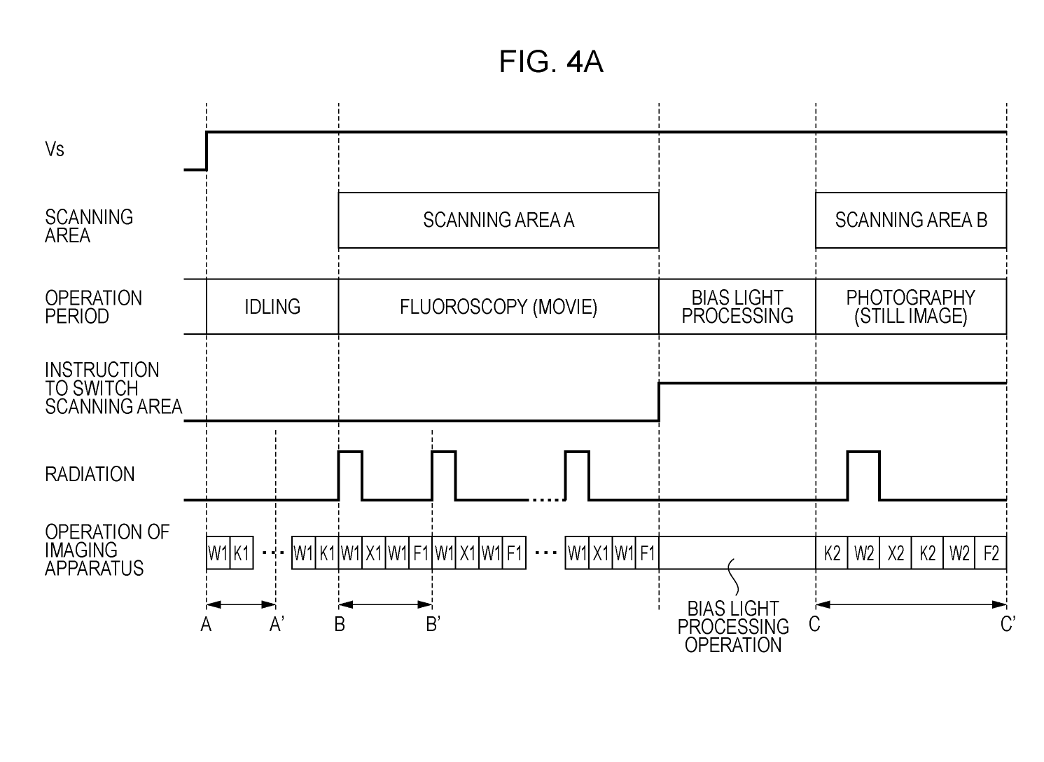

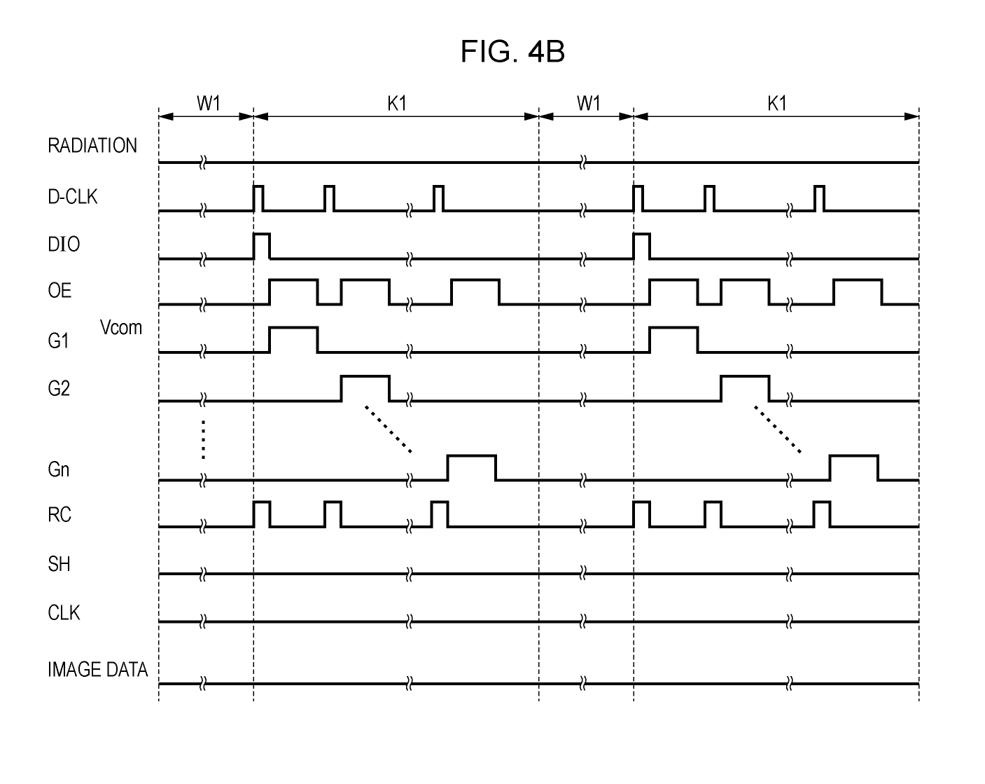

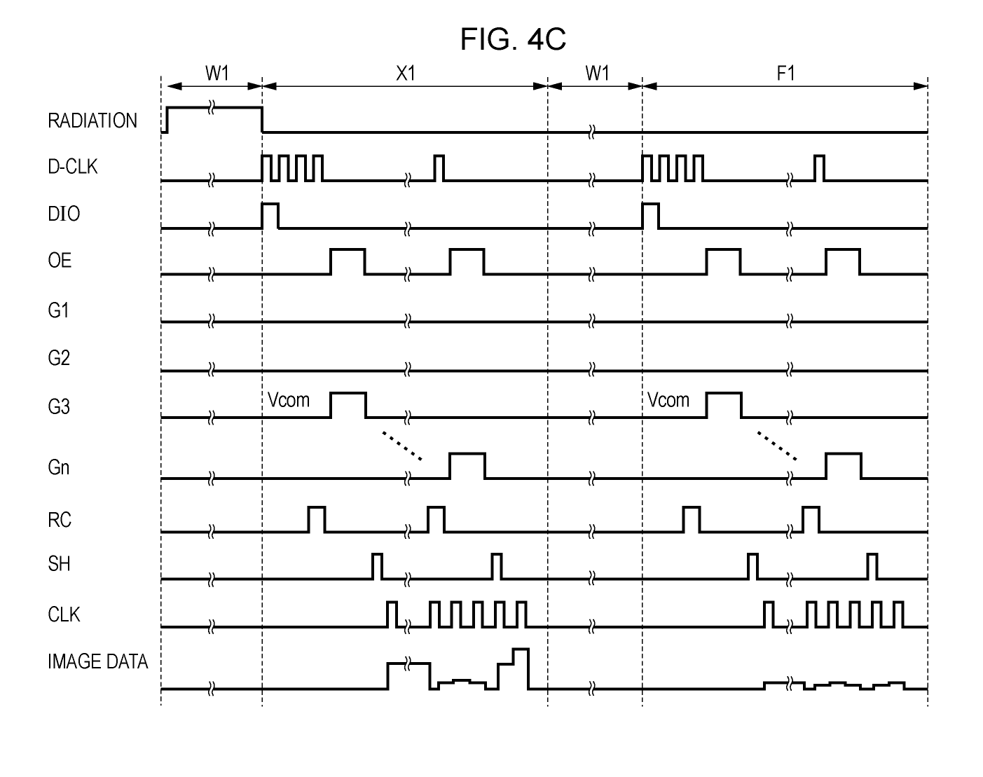

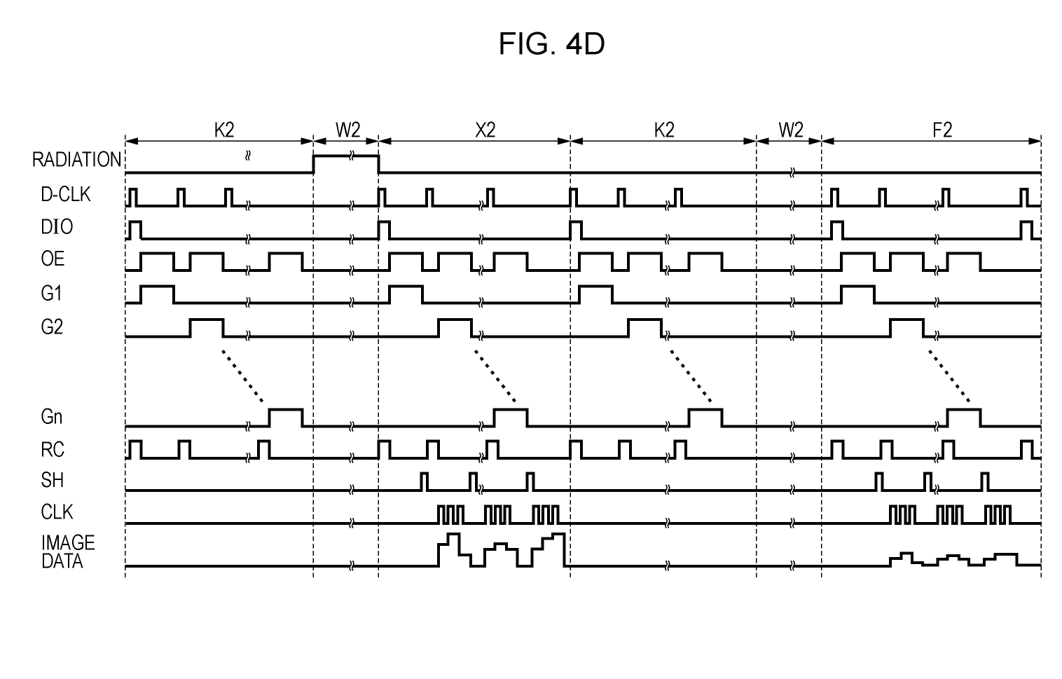

Next, imaging operations of the imaging system of the present invention will be described with reference to FIGS. 4A to 4D. Referring to FIG. 4A, upon supply of the bias voltage Vs to the conversion element 201, the imaging apparatus 100 performs an idling operation during an idling period. In the idling operation, at least an initialization operation K1 is repeated multiple times in order to stabilize the variation in characteristics of the FPD 104, caused by the start of the supply of the bias voltage Vs. The initialization operation is an operation to apply an initial bias before an accumulation operation to the conversion element to initialize the conversion element. In FIG. 4A, a pair of operations including an accumulation operation W1 and the initialization operation K1 is repeated multiple times as the idling operation.

FIG. 4B is a timing chart illustrating the operation of the imaging apparatus during a period A-A′ in FIG. 4A. Referring to FIG. 4B, in the accumulation operation W1, the non-conductive voltage Vss is applied to the switch element 202 with the bias voltage Vs applied to the conversion element 201 to set the switch elements in all of the pixels to the non-conductive state. In the initialization operation K1, the integration capacitor in the integration amplifier 203 and the signal line Sig are reset by the reset switch and the conductive voltage Vcom is applied from the drive circuit 102 to the drive line G1 to set the switch elements T11 to T1m in the first line to the conductive state. Setting the switch elements to the conductive state causes the conversion elements to be initialized. Although the electric charge of each conversion element is output from the corresponding switch element as the electrical signal in this state, no data corresponding to the electrical signal is output from the readout circuit 103 because the sample-and-hold circuit and the subsequent circuits are not operated in the present state. The integration capacitor and the signal line are reset again later to process the output electrical signal. However, when the data is to be used for correction, etc., the sample-and-hold circuit and the subsequent circuits may be operated in a manner similar to that of an image output operation or a dark image output operation described below. Repeating the control of the conductive state of the switch element and the resetting from the first line to the n-th line causes the detection unit 101 to be initialized. In the initialization operation, the reset switch may be kept in the conductive state to continue the resetting at least while the switch element 202 is in the conductive state. The time when the switch element 202 is in the conductive state in the initialization operation may be shorter than the time when the switch element is in the conductive state in the image output operation described below. In addition, the switch elements 202 in multiple lines may be operated to be in the conductive state simultaneously in the initialization operation. In such cases, it is possible to reduce the time required for the entire initialization operation to rapidly stabilize the variation in characteristics of the detection unit 101. The initialization operation K1 in the present embodiment is performed in a period having the same length as that of the period of the image output operation included in a fluoroscopy operation following the idling operation.

FIG. 4C is a timing chart illustrating an operation of the imaging apparatus (imaging operation) during a period B-B′ in FIG. 4A. After the idling operation is performed to set the detection unit 101 to a state in which the image capturing can be performed, the imaging apparatus 100 performs the fluoroscopy operation in which the detection unit 101 is scanned in the first scanning area A in response to the control signal from the control computer 108. The fluoroscopy operation corresponds to a first image capturing operation in which the scanning in the first scanning area A is performed. In the first image capturing operation, the image data corresponding to the first scanning area A is output from the FPD 104 via the readout circuit 103. The period during which the imaging apparatus 100 performs the fluoroscopy operation is called a fluoroscopy period. During the fluoroscopy period, the imaging apparatus 100 performs the accumulation operation W1 performed in a period corresponding to the irradiation time in order to cause the conversion element 201 to generate the electric charge in response to the emitted radiation and an image output operation X1 in which image data is output on the basis of the electric charge generated in the accumulation operation W1. As shown in FIG. 4C, in the image output operation in the present embodiment, the control unit 106 supplies the control signal D-CLK corresponding to the number of lines corresponding only to the second scanning area to the drive circuit 102 with the control signal OE in a Lo state. Accordingly, the conductive voltage Vcom is not supplied from the drive circuit 102 to the drive lines G1 and G2 and, thus, the first and second lines corresponding to the second scanning area are not scanned. Then, after the integration capacitor and the signal line are reset, the control unit 106 sets the control signal OE to a Hi state and supplies the control signal D-CLK corresponding to the number of lines corresponding to the first scanning area to the drive circuit 102. Accordingly, the conductive voltage Vcom is applied from the drive circuit 102 to the drive line G3 to set the switch elements T31 to T3m in the third line to the conductive state. As a result, the electrical signal based on the electric charge generated in conversion elements S31 to S3m in the third line is supplied to each signal line. Each of the electrical signals output in parallel through the respective signal lines Sig is amplified in the integration amplifier 203 and the variable amplifier 204 in each amplifier circuit 207.

The amplified electrical signals are held in parallel in the sample-and-hold circuits 205 in the respective amplifier circuits 207. The sample-and-hold circuits 205 are operated in response to the control signal SH. After the electrical signals are held, the integration capacitors and the signal lines are reset. After the resetting, the conductive voltage Vcom is applied to the drive line G4 in the fourth line, as in the third line, to set the switch elements T41 to T4m in the fourth line to the conductive state. During the period in which the switch elements T41 to T4m in the fourth line are set to the conductive state, the multiplexer 208 sequentially outputs the electrical signals held in the sample-and-hold circuits 205. As a result, the electrical signals read out from the pixels in the third line in parallel are converted into a serial image signal and the serial image signal is output. The A/D converter 210 converts the image signal into image data corresponding to one line and outputs the image data resulting from the conversion. Performing the above operation for every line from the third line to the n-th line causes the image data corresponding to one frame to be output from the imaging apparatus.

In addition, it should be noted that in the present embodiment, the imaging apparatus 100 performs the accumulation operation W1 during the image capturing operation that is performed in a period having the same length as that of the period of the accumulation operation W1 during the idling operation in order to cause the conversion element 201 to generate the electric charge in a dark state in which the emission of the radiation is not performed and a dark image output operation F1 in which dark image data is output on the basis of the electric charge generated in the accumulation operation W1. In the dark image output operation F1, an operation similar to the image output operation X1 is performed in the imaging apparatus 100. The time resulting from addition of the time when the accumulation operation is performed to the time resulting from subtraction of the time when each switch element is in the conductive state from the time when the image output operation is performed is called an “accumulation time”. The time when each switch element is in the conductive state is called a “scanning time”. The time when one set of image capturing operations including the accumulation operation, the image output operation, the accumulation operation, and the dark image output operation is performed is called a “frame time” and a reciprocal of the frame time is called a “frame speed”.

Although the pixels in the first and second lines are not scanned in the present embodiment, the present invention is not limited to this scanning mode. For example, all the second pixels corresponding to the pixels in the first and second lines may be simultaneously scanned or the second pixels may be scanned in a scanning period that is shorter than a scanning period in which the first pixels are scanned. In other words, the scanning may be performed so that the normal image capturing operation is not performed to the second pixels during the first image capturing operation. Although the pixels in the second scanning area are sequentially scanned in the initialization operation K1 in FIG. 4B, the present invention is not limited to this scanning mode and the scanning may be performed in a manner similar to that of the image output operation X1.

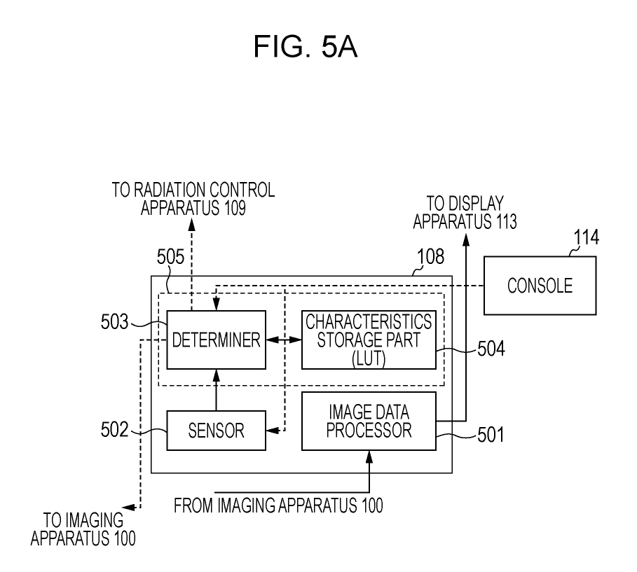

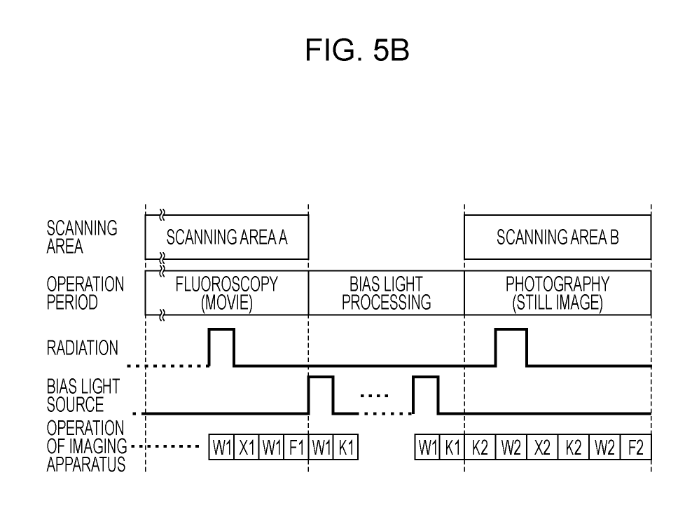

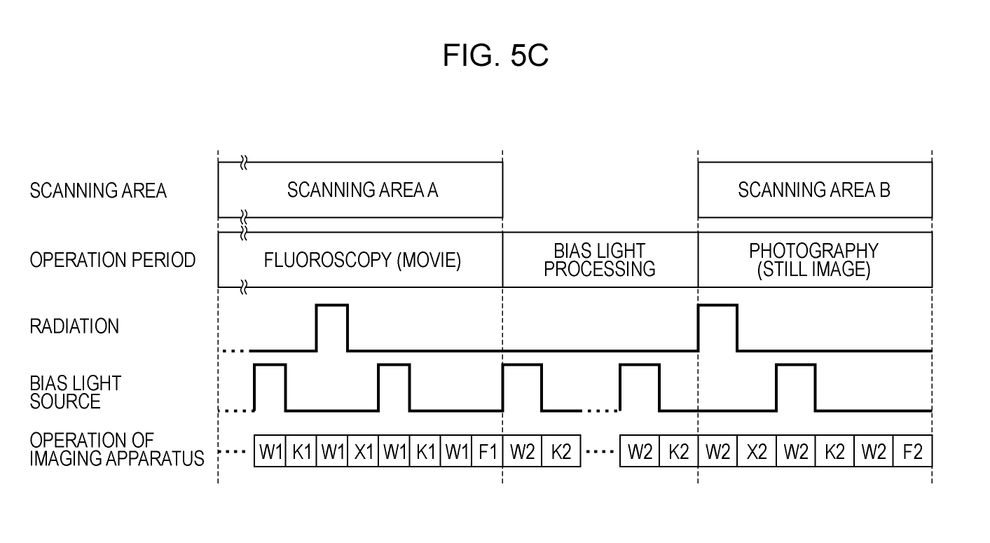

Next, the imaging apparatus 100 performs the bias light processing operation in response to an instruction received from the control computer 108. More specifically, when an operator uses the console 114 to indicate that a scanning area should be switched, the control computer 108 sends a signal to control unit 106, which in turn controls the bias light source 115 to emit bias light during a bias light processing operation shown in FIG. 4A. The period when the bias light processing operation is performed is called a bias light processing period. The bias light processing operation will be described in detail below with reference to FIG. 5.

FIG. 4D is a timing chart illustrating the operation of the imaging apparatus during a period C-C′ in FIG. 4A. After the bias light processing operation, the imaging apparatus 100 performs a photography operation (capturing of still images) in which the detection unit 101 is scanned in the second scanning area B larger than the first scanning area A. The photography operation corresponds to a second image capturing operation in which the scanning in the second scanning area B is performed. In the second image capturing operation, the image data corresponding to the second scanning area B is output from the FPD 104 via the readout circuit 103. The period in which the imaging apparatus 100 performs the photography operation is called a photography period. During the photography period, the imaging apparatus 100 performs an accumulation operation W2 performed in a period corresponding to the irradiation time in order to cause the conversion element to generate the electric charge in response to the emitted radiation and an image output operation X2 in which image data is output on the basis of the electric charge generated in the accumulation operation W2. As shown in FIG. 4D, although the accumulation operation W2 in the present embodiment is similar to the accumulation operation W1, the accumulation operation W2 is differentiated from the accumulation operation W1 because the period of the accumulation operation W2 is longer than that of the accumulation operation W1. In addition, although the image output operation X2 is similar to the image output operation X1 except that the first and second lines are scanned in the same manner as in the third line and the subsequent lines, the image output operation X2 is differentiated from the image output operation X1 because the period of the image output operation X2 is longer than that of the image output operation X1 in the present embodiment. However, the accumulation operation W2 may be performed in a period having the same length as that of the period of the accumulation operation W1 and the image output operation X2 may be performed in a period having the same length as that of the period of the image output operation X1. Furthermore, the imaging apparatus 100 performs the accumulation operation W2 performed in a period having the same length as that of the period of the accumulation operation W2 before the image output operation X2 in order to cause the conversion element to generate the electric charge in the dark state in which the radiation is not emitted and a dark image output operation F2 in which dark image data is output on the basis of the electric charge generated in the accumulation operation W2.

In the dark image output operation F2, an operation similar to the image output operation X2 is performed in the imaging apparatus 100. In addition, in the present embodiment, the imaging apparatus 100 performs an initialization operation K2 before each accumulation operation W2. Although the initialization operation K2 is similar to the initialization operation K1 described above in reference to FIG. 4B, the initialization operation K2 is differentiated from the initialization operation K1 because the period of the initialization operation K2 is longer than that of the initialization operation K1 in the present embodiment. However, the initialization operation K2 may be performed in a period having the same length as that of the period of the initialization operation K1.

The difference in level caused by the switching of scanning areas and how the embodiments of the present invention address such difference will now be described. Specifically, the inventor herein has found that a dark time output from the flat panel detector depends on the scanning history of the pixels, and that selectively reading out predetermined lines of the flat panel detector allows the dark time output to be minimized and in some instances to be entirely avoided. To that end, it has been considered that the dark time output depends on the amount of integration of the accumulation times since the bias voltage has been applied to the conversion element in the flat panel detector. The image capturing operation is performed in the first scanning area A in the first image capturing operation in the present embodiment. Accordingly, the image capturing operation is performed multiple times to the first pixels included in the first scanning area A, and the dark time output components accumulated during the accumulation operation are not completely output in each output operation and remains in the pixels. The components remaining in the pixels correspond to the scanning history of the pixels. In contrast, the normal image capturing operation is not performed to the second pixels that are not included in the first scanning area A but are included in the second scanning area B in the first image capturing operation. This is because, for example, the accumulation operation is constantly performed to the second pixels, all the second pixels in the multiple lines, which are not included in the first scanning area A but are included in the second scanning area B, are scanned at one time, or the output operation of the second pixels is performed in a scanning period shorter than that of the first pixels. In such cases, the accumulation time of the first pixels becomes different from that of the second pixels. For example, when the output operation of the second pixels is performed in a scanning period shorter than that of the first pixels, the amount of integration of the accumulation times during the first image capturing operation for the first pixels becomes smaller than that for the second pixels. In addition, since the amount of integration of the radiation in the first image capturing operation depends on the time of the first image capturing operation, the amount of integration of the radiation in the first image capturing operation depends on the amount of integration of the accumulation times. The amount of remaining electric charge causing the dark time output is varied due to the integral dose of the radiation. As a result, a difference occurs between the dark time output of the first scanning area and the dark time output of the second scanning area and the difference in the dark time output is displayed as the difference in level. Particularly, the difference in the dark time output between the first scanning area and the second scanning area is increased with the increasing period of the fluoroscopy operation and, thus, the difference in level becomes more distinct. As described above, the dark time output from the flat panel detector depends on the amount of integration of the accumulation times, which is the scanning history of the pixels. Consequently, the inventor herein has found that a difference in the dark time output occurs between the areas that are subjected to the scanning in the image capturing in the flat panel detector and the areas that are not subjected to the scanning in the image capturing in the flat panel detector to cause the difference in level, which is an image artifact caused by the scanning area.

The inventor herein has also found that the bias light processing operation described below can be performed to reduce the difference in level, which is an image artifact caused by the scanning area. The bias light source 115 irradiates the detection unit 101 in flat panel detector 104 with the bias light during a period between the first image capturing operation and the second image capturing operation in response to the switching from the first scanning area A to the second scanning area B. However, if the difference in the amount of dark time output between the first pixels and the second pixels is smaller than a predetermined threshold value in the switching of the scanning area, the difference in the amount of dark time output is not recognized as the difference in level. Particularly, it is effective to set the threshold value in consideration of the random noise of the entire image and the visual performance of a person, who is an observer of the image obtained with the first image capturing operation. If the difference in the amount of dark time output, which is the amount of image artifact, is not larger than, for example, 1/10 of the effective value of the random noise of the entire image data, the difference in level, which is an image artifact, is not recognized in the image by the observer because of the visual performance of the person.

Accordingly, the control computer 108 is specifically configured to calculate the amount of image artifact that can be caused between the areas in the switching of the scanning area on the basis of information about the amount of integration of the accumulation times in the first image capturing operation. Then, it is determined whether the bias light processing operation is to be performed on the basis of the calculated amount of image artifact and the predetermined threshold value. If it is determined that the bias light processing operation is to be performed, the control computer 108 supplies a control signal to the control unit 106 indicating that the bias light processing operation is to be performed. The control unit 106, which receives the control signal, controls the operations of the bias light source 115 and the FPD 104 in response to the control signal. If it is determined that the bias light processing operation is not to be performed, the control computer 108 supplies a control signal indicating that the bias light processing operation is not to be performed to the control unit 106. The control unit 106, which receives the control signal, controls the operation of the FPD 104 in response to the control signal and causes the bias light source 115 not to operate.

An electro luminescent (EL) panel or a light emitting diode (LED) array in which multiple LED elements are arranged in a matrix may be used as the bias light source 115. In one embodiment, the bias light source 115 may include a matrix of n lines by m columns, where n and m have a one-to-one correspondence with the number of lines and columns included in the detection unit 101. That is, the bias light source 115 may have multiple light emitting elements arranged in a matrix having the same number of lines and columns as the matrix of pixels included in the detection unit 101. In alternative embodiments, the bias light source 115 may include a matrix of n lines by m columns, where n and m have a one-to-four correspondence with the number of lines and columns included in the detection unit 101. That is, the bias light source 115 may have a matrix of light emitting elements arranged in a manner so that each bias light emitting element corresponds to four pixels included in the detection unit 101.

Next, the configuration in which a determination process of the present invention is performed and a specific determination process will now be described with reference to FIG. 5A. The control computer 108 includes an image data processor 501, a sensor 502, a determiner 503, and a characteristics storage part 504. The characteristics storage part 504 stores the amount of integration of the accumulation times in the first image capturing operation, the amount of image artifact corresponding to the scanning pattern in the second scanning area in the first image capturing operation, and information about the predetermined threshold value. Specifically, the scanning is performed to the second pixels included in the second scanning area B in the following three patterns. In the first scanning pattern, the accumulation operation is constantly performed to the second pixel. In the second scanning pattern, all of the multiple second pixels or the second pixels in the multiple lines are simultaneously scanned. In the third scanning pattern, the output operation of the second pixels is performed in a scanning period that is shorter than that of the pixels in the first scanning area. The amounts of image artifact in the three respective patterns are measured in advance in association with the amount of integration of the accumulation times and are stored in the characteristics storage part 504. A lookup table in which such data is stored is preferably used as the characteristics storage part 504. In the present invention, the determiner 503 and the characteristics storage part 504 are collectively referred to as an arithmetic processing unit 505.

The image data output from the imaging apparatus 100 is subjected to the image processing in the image data processor 501 and is transmitted to the display apparatus 113. At this time, the sensor 502 calculates the accumulation time for every scanning area from the operation time of each frame and accumulates the calculated accumulation times. The sensor 502 adds up the accumulated accumulation times in units of frames to generate the information about the amount of integration of the accumulation times in each scanning area in the first image capturing operation. For example, the information about the amount of integration of the accumulation times in the first image capturing operation may be based on information about the image capturing conditions in the first image capturing operation acquired from the console 114. The sensor 502 supplies the generated information about the amount of integration of the accumulation times to the determiner 503.

The determiner 503 determines whether the bias light processing operation is to be performed on the basis of the information about the amount of integration of the accumulation times supplied from the sensor 502, the amount of image artifact, and the predetermined threshold value. If it is determined that the bias light processing operation is to be performed, the arithmetic processing unit 505 supplies the control signal indicating that the bias light processing operation is to be performed to the control unit 106. The control unit 106, which receives the control signal, controls the operations of the bias light source 115 and the FPD 104 in response to the control signal. If it is determined that the bias light processing operation is not to be performed, the arithmetic processing unit 505 supplies the control signal indicating that the bias light processing operation is not to be performed to the control unit 106. Here, the integral dose of the radiation is varied with the amount of integration of the accumulation times. As a result, the amount of electric charge remaining in the conversion element, which causes the dark time output, can be varied to vary the sensitivity of the conversion element. In such a case, the quantity of bias light necessary for the bias light processing operation is varied. Accordingly, it is desirable that the control unit 106 determine the quantity of light emitted from the bias light source on the basis of the amount of integration of the accumulation times and control the operation of the bias light source so that the determined quantity of light is emitted. This allows the bias light processing operation to be performed with a small quantity of light, thereby reducing the power consumption of the bias light source. The control unit 106, which receives the control signal, controls the operation of the FPD 104 in response to the control signal and causes the bias light source 115 not to operate. Although the control computer 108 determines whether the bias light processing operation is to be performed in the present embodiment, the present invention is not limited to this. The control unit 106 in the imaging apparatus 100 may determine whether the bias light processing operation is to be performed in response to the control signal transmitted from the control computer.

Next, exemplary bias light processing operations of the present embodiment will now be described with reference to FIGS. 5B and 5C. In the bias light processing operations of the present invention, the bias light source 115 irradiates the FPD 104 with the bias light. After the emission of the bias light, the FPD 104 initializes the conversion element. It was found that performing a pair of the emission of the bias light and the initialization operation of the conversion element multiple times further reduces the difference in level. The bias light processing operation in which the pair of the emission of the bias light and the initialization operation of the conversion element is performed once or multiple times can be performed to prevent a reduction in image quality caused by the difference in level that can occur in an acquired image as the result of the switching of the scanning area.

In the bias light processing operation shown in FIG. 5B, the bias light source 115 emits the bias light in accordance with the emission of the radiation in the fluoroscopy operation performed before the switching of the scanning area, described above with reference to FIG. 4C. Then, the FPD 104 performs a pair of the accumulation operation W1 in the fluoroscopy operation and the initialization operation K1 once or multiple times. Specifically, the FPD 104 performs the pair of the accumulation operation W1 corresponding to the fluoroscopy operation performed before the switching of the scanning area and the initialization operation K1 once or multiple times. With the bias light processing operation in FIG. 5B, the time necessary for the operation is decreased to improve the responsiveness of the apparatus. However, when the initialization operation performed in the bias light processing operation does not correspond to the image capturing operation before the switching of the scanning area and is performed in a period having a length different from that of the period of the initialization operation performed in the image capturing operation before the switching of the scanning area, the stability of the characteristics of the conversion element in the accumulation operation in the image capturing operation can be degraded. As a result, image data having a large amount of image artifact can possibly acquired.

In the bias light processing operation shown in FIG. 5C, the bias light source 115 emits the bias light in accordance with the emission of the radiation in the photography operation performed after the switching of the scanning area, described above with reference to FIG. 4D. Then, the FPD 104 performs a pair of the accumulation operation W2 in the photography operation performed after the switching of the scanning area and the initialization operation K2 once or multiple times. Specifically, the FPD 104 performs the pair of the accumulation operation W2 corresponding to the photography operation performed after the switching of the scanning area and the initialization operation K2 once or multiple times. Performing the bias light processing operation in accordance with the operation included in the operations before the image output operation in the image capturing operation after the switching in the above manner allows the characteristics of the conversion element in the accumulation operation W2 in the image capturing operation to be stabilized to acquire excellent image data having a reduced amount of image artifact. In FIG. 5C, in the fluoroscopy operation, the emission of the bias light in accordance with the accumulation operation W1 and the initialization operation K1 are performed before the pair of the accumulation operation W1 and the image output operation X1 and the pair of the accumulation operation W1 and the dark image output operation F1. In addition, in the photography operation, the emission of the bias light in accordance with the accumulation operation W2 and the initialization operation K2 are performed before the pair of the accumulation operation W2 and the dark image output operation F2. Particularly, in the photography operation, the emission of the bias light in the bias light processing operation and the initialization operation K2 are performed before the emission of the radiation. Accordingly, performing the emission of the bias light and the initialization operation K2 before the pair of the accumulation operation W2 and the dark image output operation F2 allows the pair of the accumulation operation W2 and the image output operation X2 to be matched with the pair of the accumulation operation W2 and the dark image output operation F2. As a result, it is possible to match the effect of the dark output of the radiation on the image data with the effect of the dark output of the radiation on the dark image data, thereby acquiring excellent image data having a reduced amount of image artifact.

As described above, the bias light processing operation can be performed before start of the image capturing operation after the scanning area is switched to reduce the image artifact (difference in level) that can occur in a acquired image and that is affected by the scanning area, thereby preventing a significant reduction in image quality.

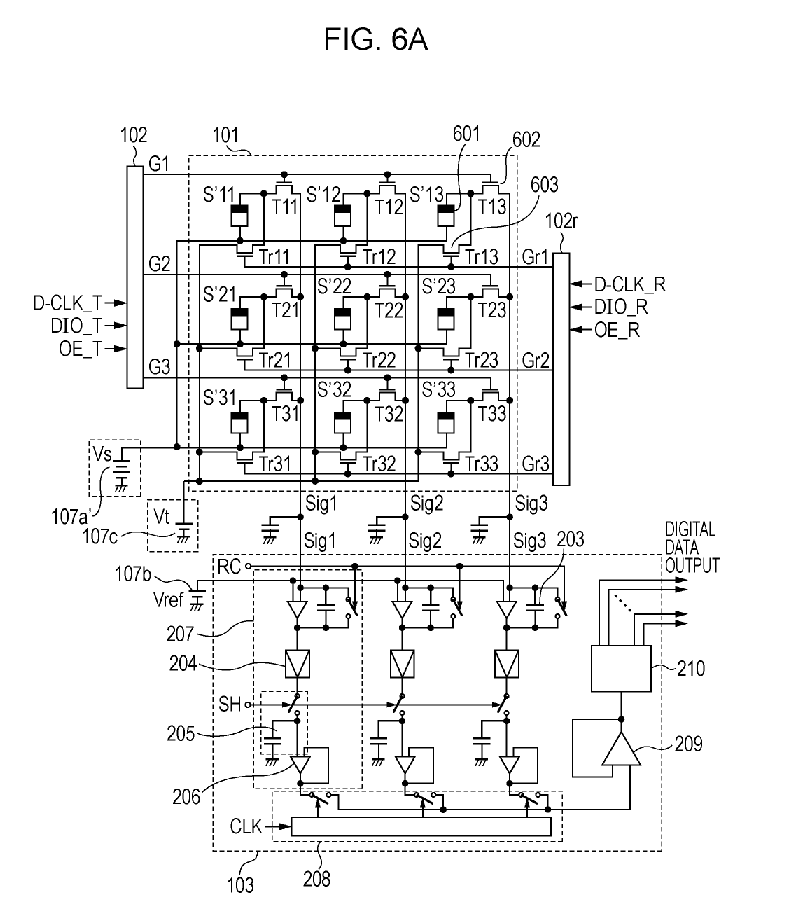

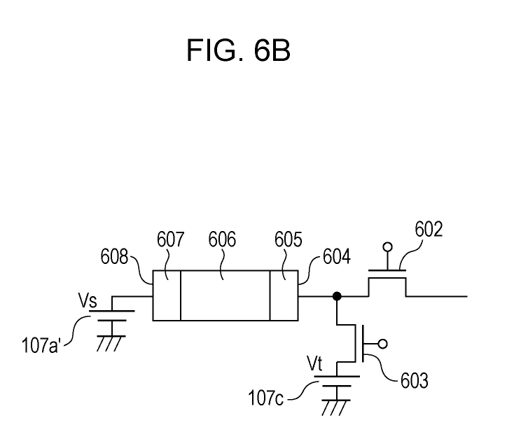

Although the PIN photodiode is used in the conversion element 201 in the present embodiment, the present invention is not limited to the PIN photodiode. An imaging apparatus using pixels in which a photoelectric transducer having a metal insulator semiconductor (MIS) structure is used as a MIS-type conversion element in a conversion element 601 and a refresh switch element 603 is provided, in addition to an output switch element 602, may be used, as shown in FIGS. 6(a) and 6(b). In FIG. 6(a), one of the main terminals of the refresh switch element 603 is electrically connected to a first electrode 604 of the conversion element 601 and to one of the two main terminals of the output switch element 602. The other of the main terminals of the refresh switch element 603 is electrically connected to a refresh power supply 107c included in the power supply unit 107 via a common line. The control terminals of the multiple refresh switch elements 603 in the line direction are commonly electrically connected to a refresh drive line Gr. A drive signal is supplied from a refresh drive circuit 102r to the refresh switch elements 603 in each line through the refresh drive line Gr. As shown in FIG. 6(b), in the conversion element 601, a semiconductor layer 606 is provided between the first electrode 604 and a second electrode 608, an insulating layer 605 is provided between the first electrode 604 and the semiconductor layer 606, and an impurity semiconductor layer 607 is provided between the semiconductor layer 606 and the second electrode 608. The second electrode 608 is electrically connected to a bias power supply 107a′ via the bias line Bs. The bias voltage Vs is supplied from the bias power supply 107a′ to the second electrode 608 in the conversion element 601 and the reference voltage Vref is supplied to the first electrode 604 in the conversion element 601 through the output switch element 602 to perform the accumulation operation in the conversion element 601, as in the conversion element 201. In the fluoroscopy operation and the photography operation, a refresh voltage Vt is supplied to the first electrode 604 through the refresh switch element 603 and the conversion element 601 is refreshed with a bias |Vs-Vt|. The same reference numerals are used in FIGS. 6(a) and 6(b) to identify the same components in FIG. 2. A detailed description of such components is omitted herein.

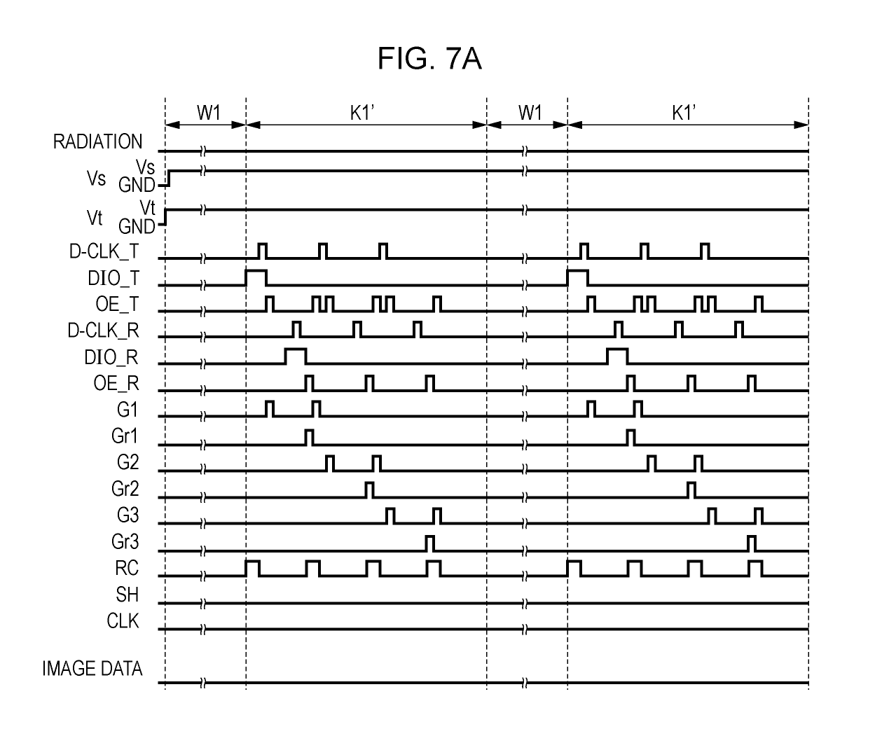

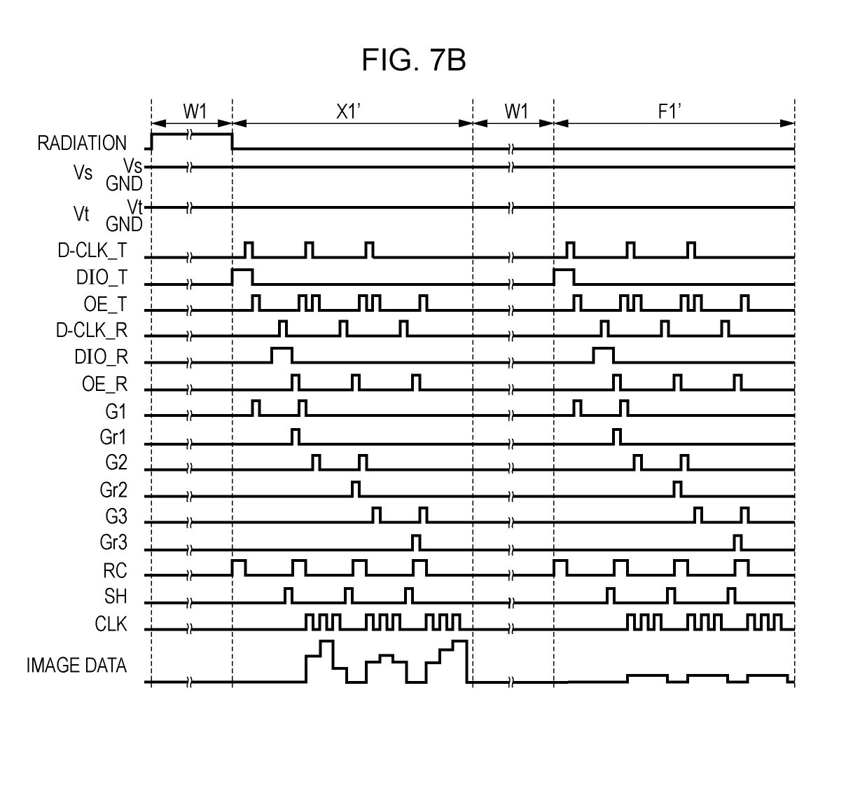

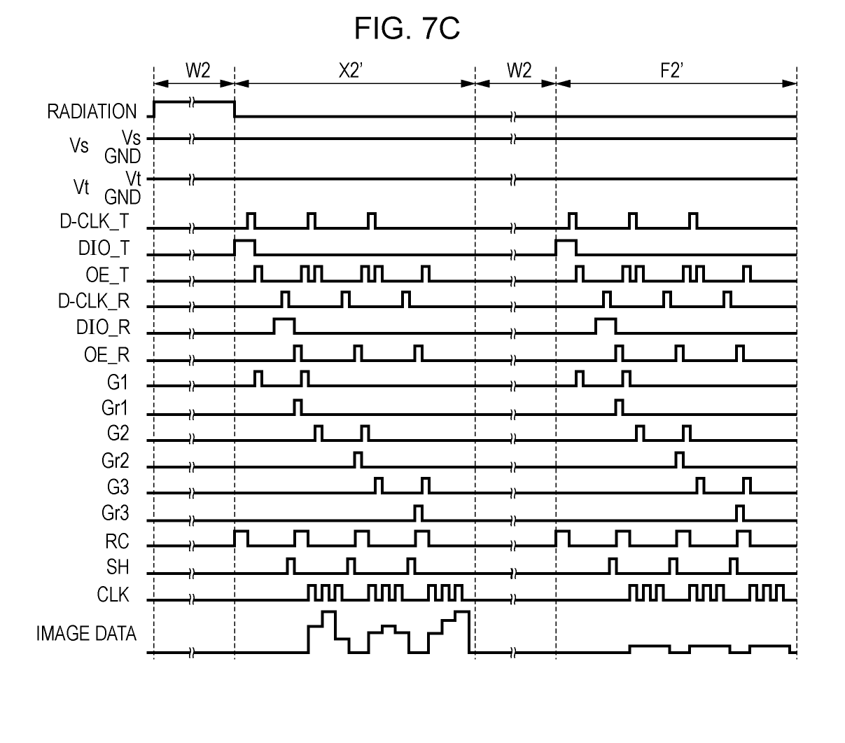

The operations of the imaging apparatus in FIG. 6 are shown in FIGS. 7A to 7C. FIG. 7A is a timing chart illustrating the operation of the imaging apparatus during the period A-A′ in FIG. 4A. FIG. 7B is a timing chart illustrating the operation of the imaging apparatus during the period B-B′ in FIG. 4A. FIG. 7C is a timing chart illustrating the operation of the imaging apparatus during the period C-C′ in FIG. 4A. An initialization operation K1′, an image output operation X1′, and a dark image output operation F1′ are performed, instead of the initialization operation K1, the image output operation X1, and the dark image output operation F1, respectively, in the first embodiment shown in FIG. 4A. In addition, an image output operation X2′ and a dark image output operation F2′ are performed, instead of the image output operation X2 and the dark image output operation F2, respectively, in the first embodiment shown in FIG. 4A. The remaining operations are similar to the ones in FIG. 4A. A detailed description of such operations is omitted herein.

The embodiments of the present invention may be realized by, for example, a program executed by a computer included in the control unit 106. A unit to supply the program to the computer, for example, a computer-readable recording medium, such as a compact disc-read only memory (CD-ROM), having the program recorded therein or a communication medium, such as the Internet, over which the program is transmitted is also applicable as an embodiment of the present invention. In addition, the program is also applicable as an embodiment of the present invention. The program, the recording medium, the communication medium, and the program product are within the scope of the present invention. A combination easily supposed from the present embodiments is also within the scope of the present invention.

According to the present invention, the drive operation of the FPD allows ghost (difference in level) that can occur in an acquired image and that is affected by the scanning area to be reduced to prevent a considerable reduction in image quality.

While the present invention has been described with reference to exemplary embodiments, it is to be understood that the invention is not limited to the disclosed exemplary embodiments. In particular, in the present invention, the radiation includes not only alpha rays, beta rays, and gamma rays, which are beams made of particles (including photons) emitted due to radiation damage, but also beams, such as X rays, particle beams, and cosmic rays, having the energies of at least the same level as those of the alpha rays, the beta rays, and the gamma rays. Accordingly, the scope of the following claims is to be accorded the broadest interpretation so as to encompass all modifications and equivalent structures and functions.

This application claims the benefit of International Application No. PCT/JP2009/070201, filed Dec. 1, 2009, which is hereby incorporated by reference herein in its entirety.

### REFERENCE SIGNS LIST

100 imaging apparatus
101 detection unit
102 drive circuit
103 readout circuit
104 flat panel detector
105 signal processing unit
106 control unit
107 power supply unit
108 control computer
109 radiation control apparatus
110 radiation generating apparatus
111 radiation source
112 irradiation field limiting mechanism
113 display apparatus
114 console
115 bias light source

1. An imaging system comprising:
an imaging apparatus including
a detector in which a plurality of pixels each including a conversion element that converts radiation or light into an electric charge are arranged in a matrix and which performs an image capturing operation to output image data corresponding to radiation or light that is emitted;
a bias light source that irradiates the detector with bias light different from the radiation or the light; and
a control unit that controls operations including the image capturing operation of the detector and an operation of the bias light source; and
a control computer that controls the imaging apparatus,
wherein the image capturing operation includes a first image capturing operation in which the detector is scanned in a first scanning area corresponding to part of the plurality of pixels to output image data in the first scanning area and a second image capturing operation in which the detector is scanned in a second scanning area larger than the first scanning area to output image data in the second scanning area,
wherein the control computer determines the operation of the bias light source on the basis of information about the amount of integration of accumulation times in the first image capturing operation and supplies a control signal based on the determined operation of the bias light source to the control unit, and
wherein the control unit controls the operation of the bias light source so that the bias light source emits the bias light on the basis of the control signal during a period between the first image capturing operation and the second image capturing operation in accordance with switching from the first scanning area to the second scanning area.

2. The imaging system according to claim 1,
wherein the control computer determines whether the emission of the bias light by the bias light source is to be performed on the basis of the information about the amount of integration of the accumulation times, supplies a control signal to perform the emission of the bias light to the control unit if the control computer determines that the emission of the bias light is to be performed, and supplies a control signal not to perform the emission of the bias light to the control unit if the control computer determines that the emission of the bias light is not to be performed.

3. The imaging system according to claim 2,
wherein the control computer includes a characteristics storage part, a sensor, and a determiner,
wherein the storage part stores information about the amount of image artifact corresponding to the amount of integration of the accumulation times in the first image capturing operation and information about a predetermined threshold value,
wherein the sensor supplies the information about the amount of integration of the accumulation times in the first image capturing operation to the determiner, and
wherein the determiner determines whether the emission of the bias light is to be performed on the basis of the information about the amount of integration of the accumulation times supplied from the sensor and the information about the amount of image artifact and the information about the predetermined threshold value stored in the storage part.

4. The imaging system according to claim 3,
wherein the information about the amount of image artifact is acquired in advance in accordance with a scanning pattern in the second scanning area in the first image capturing operation and the amount of integration of the accumulation times, and
wherein the information about the predetermined threshold value is set in advance so that the amount of image artifact is not larger than 1/10 of the effective value of a random noise of the image data.

5. The imaging system according to claim 3, further comprising:
a console that supplies information about an image capturing condition in the first image capturing operation to the control computer,
wherein the sensor acquires the information about the amount of integration of the accumulation times in the first image capturing operation from the console.

6. The imaging system according to claim 1,
wherein the control unit controls the operation of the detector so that the detector performs an initialization operation to initialize the conversion element after the emission of the bias light.

7. The imaging system according to claim 6,
wherein each of the pixels further includes a switch element that outputs an electrical signal corresponding to the electric charge,
wherein the detector includes a detection unit in which the pixels are arranged in a matrix, a drive circuit that controls a conductive state of the switch element to drive the detection unit, and a readout circuit that outputs the electrical signal supplied from the detection unit through a signal line connected to the switch element as image data,
wherein the readout circuit includes a reset switch that resets the signal line, and
wherein the control unit controls the drive circuit and the reset switch so that the detector performs the initialization operation to initialize the conversion element after the emission of the bias light.

8. The imaging system according to claim 6,
wherein the conversion element is a metal insulator semiconductor (MIS)-type conversion element,
wherein each of the pixels further includes a first switch element that outputs an electrical signal corresponding to the electric charge and a second switch element different from the first switch element,
wherein the detector further includes a detection unit in which the pixels are arranged in a matrix, a first drive circuit that controls the conductive state of the first switch element to drive the detection unit, a readout circuit that outputs the electrical signal supplied from the detection unit through a signal line connected to the first switch element as image data, a second drive circuit that controls the conductive state of the second switch element, and a power supply unit including a reference power supply that applies a reference voltage to one electrode of the conversion element through the first switch element, a refresh power supply that applies a refresh voltage to the one electrode through the second switch element, and a bias power supply that applies a bias voltage to the other electrode of the conversion element, and
wherein the detector sets the first switch element to a non-conductive state, sets the second switch element to the conductive state, applies the bias voltage to the other electrode, and applies the refresh voltage to the other electrode through the second switch element to refresh the conversion element.

9. An imaging apparatus comprising:
a detector in which a plurality of pixels each including a conversion element that converts radiation or light into an electric charge are arranged in a matrix and which performs an image capturing operation to output image data corresponding to radiation or light that is emitted;
a bias light source that irradiates the pixels with bias light different from the radiation or the light; and
a control unit that controls operations including the image capturing operation of the detector and an operation of the bias light source,
wherein the image capturing operation includes a first image capturing operation in which the detector is scanned in a first scanning area corresponding to part of the plurality of pixels to output image data in the first scanning area and a second image capturing operation in which the detector is scanned in a second scanning area larger than the first scanning area to output image data in the second scanning area, and
wherein the control unit controls the operation of the bias light source so that the bias light source emits the bias light on the basis of a control signal based on information about the amount of integration of accumulation times in the first image capturing operation during a period between the first image capturing operation and the second image capturing operation in accordance with switching from the first scanning area to the second scanning area.

10. A method of controlling an imaging apparatus that includes a detector in which a plurality of pixels each including a conversion element that converts radiation or light into an electric charge are arranged in a matrix and which performs an image capturing operation to output image data corresponding to radiation or light that is emitted and a bias light source irradiating the pixels with bias light different from the radiation or the light and that controls operations including the image capturing operation of the detector and an operation of the bias light source, the method comprising the steps of:
performing a first image capturing operation in which the detector is scanned in a first scanning area corresponding to part of the plurality of pixels to output image data in the first scanning area;
determining the operation of the bias light source on the basis of information about the amount of integration of accumulation times in the first image capturing operation; and
emitting the bias light on the basis of the determined operation of the bias light source during a period between the first image capturing operation and a second image capturing operation in which the detector is scanned in a second scanning area larger than the first scanning area to output image data in the second scanning area in accordance with an instruction to switch from the first scanning area to the second scanning area in order to perform the second image capturing operation.

11. A program causing a computer to control an imaging apparatus that includes a detector in which a plurality of pixels each including a conversion element that converts radiation or light into an electric charge are arranged in a matrix and which performs an image capturing operation to output image data corresponding to radiation or light that is emitted and a bias light source irradiating the pixels with bias light different from the radiation or the light and that controls operations including the image capturing operation of the detector and an operation of the bias light source, the program comprising the steps of:
performing a first image capturing operation in which the detector is scanned in a first scanning area corresponding to part of the plurality of pixels to output image data in the first scanning area;
determining the operation of the bias light source on the basis of information about the amount of integration of accumulation times in the first image capturing operation; and
emitting the bias light on the basis of the determined operation of the bias light source during a period between the first image capturing operation and a second image capturing operation in which the detector is scanned in a second scanning area larger than the first scanning area to output image data in the second scanning area in accordance with an instruction to switch from the first scanning area to the second scanning area in order to perform the second image capturing operation.
